(19) United States
(12) Patent Application Publication
(10) Pub. No.: US 2011/0313306 A1
(43) Pub. Date: Dec. 22, 2011

(54) METHOD AND APPARATUS FOR MONITORING STRESS LEVELS OR SUDDEN CHANGES OF HUMOR IN HUMANS OR OTHER INDIVIDUALS IN REAL TIME BY MEANS OF VAPOR ANALYSIS

(75) Inventors: RAFAEL BORRAJO-PELAEZ, Malaga (ES); ERNESTO CRIADO-HIDALGO, Valladolid (ES); GUILLERMO VIDAL-DE-MIGUEL, Valladolid (ES)

(73) Assignee: SOCIEDAD EUROPEA DE ANALISIS DIFERENCIAL DE MOVILIDAD, Madrid (ES)

(21) Appl. No.: 13/161,662

(22) Filed: Jun. 16, 2011

Related U.S. Application Data
(60) Provisional application No. 61/355,252, filed on Jun. 16, 2010.

(51) Int. Cl. A61B 5/08

(52) U.S. Cl. 600/532

(57) ABSTRACT

A method is described to perform on-line detection of stress levels or changes in humor of individuals. The approach is particularly advantageous because analysis can be performed in real time or, at least, comprising a delay shorter than 30 s, and because it is non-invasive and non-degrading; and actually the individual under study do not need to be aware of being studied. More specifically, the present invention is based on the analysis of lactic acid and other volatiles concentrations in the vapors released by the individual (through the skin and the breath). The inventors have proved that said concentrations respond nearly instantaneously when the individual is subjected to a sudden stressful stimulus.

### CROSS REFERENCE TO RELATED APPLICATION

This application claims the benefit of priority to U.S. Provisional Patent Application No. 61/355,252, filed Jun. 16, 2010, the entire contents of which is incorporated by reference herein.

### U.S. PATENTS AND APPLICATIONS CITED

U.S. Pat. No. 4,531,056; Michael J. Labowsky, John B. Fenn, Masamichi Yamashita; Method and apparatus for the mass spectrometric analysis of solutions; Apr. 20, 1983.

U.S. 61/204,996; Improved ionizer for vapor analysis decoupling the ionization region from the analyzer; Vidal-de-Miguel G. 13 Jan. 2010

U.S. Ser. No. 11/732,770; Martinez-Lozano P., Fernandez de la Mora J.; Method for detecting volatile species of high molecular weight; Apr. 4, 2006.

U.S. Pat. No. 3,971,034; Bell, Jr.; Allan D. (Annandale, Va.), Ford; Wilson H. (Arlington, Va.), McQuiston; Charles R. (Falls Church, Va.). Physiological response analysis method and apparatus; Jul. 20, 1976.

U.S. Ser. No. 11/441,449; Humble, Charles. Quantifying psychological stress levels using voice patterns; May 25, 2006.

U.S. Pat. No. 7,451,079; Oudeyer; Pierre-Yves; Emotion recognition method and device, Jul. 12, 2002.

U.S. Ser. No. 10/660,025; Cobain et al. Psychological stress in humans, Mar. 18, 2004.

### OTHER PATENTS AND APPLICATIONS CITED

Patent application WO/2008/084403; Fernandez de la Mora G; Method and apparatus for the identification of persons based on the analysis of volatile substances of said persons in the surrounding gas; 17, Jul., 2008.

### OTHER DOCUMENTS CITED

[1] Cheng, W-H and Lee, W-J, Technology Development in Breath Microanalysis for Clinical Diagnosis. J. Lab. Clin. Med. 133, 218-228 (1999)
[2] Fenn J B, Mann M, Meng C K, Wong S F, Whitehouse C M, Electrospray ionization for mass spectrometry of large biomolecules. Science 246 (4926): 64-71, 1989
[3] Whitehouse, C. M., Levin, F., Meng, C. K. and Fenn, J. B., Proc. 34th ASMS Conf. on Mass Spectrom. and Allied Topics, Denver, 1986, p. 507.
[4] Fuerstenau, S., Kiselev, P. and Fenn, J. B., ESIMS in the Analysis of Trace Species in Gases, Proceedings of the 47th ASMS Conference on Mass Spectrometry (1999) Dallas Tex.
[5] P. Martinez-Lozano, J. Rus, G. Fernández de la Mora, M. Hernández, J. Fernández de la Mora, Secondary Electrospray Ionization of Ambient Vapors for Explosive Detection at Concentrations Below Parts Per Trillion. J. Am. Soc. Mass Spectr. Volume 20, Issue 2, February 2009, Pages 287-294.
[6] P. Martinez Lozano and J. Fernández de la Mora, Electrospray ionization of volatiles in breath, Journal of American Society of Mass Spectrometry, Volume 265, Issue 1, August 2007, pages 68-72.
[7] P. Martinez-Lozano and J. Fernández de la Mora, Direct analysis of fatty acid vapors in breath by electrospray ionization and atmospheric pressure ionization mass spectrometry, Analytical Chemistry, 2008, 80, 8210-8215
[8] P. Martinez-Lozano and J. Fernández de la Mora, On-line detection of human skin vapors, Journal of American Society of Mass Spectrometry, Volume 20, Issue 6, June 2009, pages 1060-1063.
[9] Lindinger, W., Hansel, A., Jordan, A., On-line monitoring of volatile organic compounds at pptv level by means of Proton-Transfer-Reaction Mass Spectrometry (PTR-MS). Medical applications, food control and environmental research. International Journal of Mass Spectrometry and Ion Processes. 173 (1998) 191-241.
[10] Amann, A. et al., Applications of breath gas analysis. International Journal of Mass Spectrometry 239 (2004) 227-233
[11] M. Gallagher, C. J. Wysocki, J. J. Leyden, A. I. Spielman, X. Sun and G. Preti, Analyses of volatile organic compounds from human skin, British Journal of Dermatology, 2008, 159, pp 780-791.
[12] Acree, F., Jr.; Turner, R. B.; Gouck, H. K; Beroza, M.; Smith, N. L-Lactic acid: A mosquito atractant isolated from humans. Science 1968, 161, 1346-1347.
[13] Bernier, U, R.; Kline, D. L.; Barnard, D. R.; Schreck, C. E.; Yost, R. A. Analysis of human skin emanations by gas chromatography/mass spectrometry. 2. Identification of volatile compounds that are candidate attractants for the yellow fever mosquito. (aedes aegypti). Anal. Chem. 2000, 72, 747-756
[14] Curran, A. M.; Rabin, S. 1.; Prada, P. A., Futon, KG. Comparison of the volatile organic compounds present in human odor using SPME-GC/MS. J. Chem. Ecol. 2005, 31, 1607-1619.
[15] M. McCulloch et al; Diagnostic Accuracy of Canine Scent Detection in Early- and Late-Stage Lung and Breast Cancers Integ. Cancer Therapies 5(1), 1 (2006)
[16] Lippold, O. C. J.; Oscillation in the stretch reflex arc and the origin of the rhythmical, 8-12 c/s component of physiological tremor; The Journal of Physiology; 1970, February, 206(2), 359-382.
[17] Seeman, T. E.; McEwen, B. S.; Rowe, J. W. et al; Allostatic load as a marker of cumulative biological risk: MacArthur studies of successful aging; Proceedings of the National Academy of Sciences of the United States of America; 2001, Vol. 98, no. 8, p. 4770-4775.
[18] Lupien S. J.; Gaudreau S.; Tchiteya, B. M. et al; Stress-induced declarative memory impairment healthy elderly subjects: Relationship to cortisol reactivity; Journal of Clinical Endocrinology and Metabolism; 1997, Vol. 82, no. 7, p. 2070-2075.
[19] Liu, T.; Stern, A. et al; The Isoprostanes: Novel prostaglandin-like products of the free radical catalysed peroxidation of arachidonic acid; Journal of Biomedical Science; 1999, Vol. 6, no. 4 pp 226-235.

### FIELD OF THE INVENTION

The invention relates to the problem of determining stress levels or sudden changes in humor in human or other type of individuals in real time, and comprising neither invasively nor degrading methods. More specifically, the method permits an on-line analysis of lactic acid, pyruvic acid and other volatiles from skin or breath as markers of stress levels and changes in humor of said individuals. This is done on-line sampling vapors released by the skin or breath of the individual under analysis and further on-line analyzing the chemical composition of said vapors. These said volatiles from skin or breath are then identified as stress-dependent species and thus classified as potential markers of said sudden changes in humor in human or other type of individuals.

### BACKGROUND OF THE INVENTION

The analysis of chemical species in a gas has been widely used as a source of information about the presence of air contaminants, drug analysis, food and aroma industries or even the detection of explosives in certain situations. The most important technique used with this purpose has been the GC-MS method [1], however, it was not until the arrival of Fenn and colleagues' method [U.S. 4531056, Refs. 2, 3, 4], that the vapor analysis via electrospray ionization-mass spectrometry (ESI-MS) experienced a great evolution, both in the speed of analysis, when compared with GC-MS, and the limit of detection of low volatile substances. Fenn and coworkers noted that vapors put in contact with an electrospray cloud were efficiently ionized; achieving limits of detection up to the ppb level (parts per billion). Other ionization techniques have also been used in this direction, such us Ni-63 radioactive ionization, corona discharges, etc. But all of them achieve lower sensitivities. The field of this ESI-MS technique has recently been reviewed by Martinez-Lozano, J. Fernandez de la Mora and G. Vidal [Ref. [5]; U.S. 61/204,996].

Human vapors and breath analysis art. The starting point for the present invention are two previous patents in the area [U.S. Ser. No. 11/732,770; WO/2008/084403] and the publication of three specific papers related to the analysis of human vapors in breath or in the skin [6, 7, 8] (published following the application in [U.S. Ser. No. 11/732,770]). The application in [U.S. Ser. No. 11/732,770] and the papers in [6, 7, 8] showed that when human vapors from the skin or breath are collected and later put in contact with an electrospray cloud, they are efficiently charged and can be analyzed in a mass or mobility analyzer such as mass spectrometers (MS), ion mobility spectrometer (IMS) or differential mobility analyzers (DMA). The instrumentation used permitted identifying and quantifying the low concentrations of the species present in the vapors released from the skin or from the human breath [6, 7, 8, 9, 10, 11, 12, 13, 14]. Ref. [15] also tries to make a relation between some health problems and an anomalous breath spectrum based on the evidence that dogs can identify persons suffering from some kinds of cancer. This also suggests that there must exist a numerous of highly volatile species related to some specific cancers. This assert is also supported by the high sensitivity level of Martinez-Lozano and coworkers' API-MS technique (10⁻¹² atmospheres), that approaches the detection thresholds generally achieved by dogs [5].

Many studies have been published before showing that the human breath contains a rich mixture of species [6, 7, 10]. However, the new aspect in [7, 8] was the fact that the level of the signal of the species detected and positively identified was much higher than in the background. Also, species with masses between 200 and 600 amu were positively identified. On the contrary, previous studies identified highly volatile molecules (lower molecular weight species) and with signal levels very similar to that found in the background. This aspect is important in biological studies, because high molecular weight species tend to be more specific and thus, give much more information on the biological processes in which they are involved in.

Human stress level recognition background. The first devices used with the aim of measuring human stress levels were named polygraphs. They were first used as lie detectors and monitored changes in numerous physiological parameters as heart rate, breath rate, blood pressure or even electrodermal activity. The state of art in this technology has changed very little since the beginning of the XIXth century and has the great inconvenience of being very invasive or even degrading for the individual because of the numerous probes that have to be in direct contact with the said individual.

Another technology used with the target of determining stress levels in humans is based on voice recognition. This technology first appeared in 1976 [U.S. Pat. No. 3,971,034] and is based on the discovery of vocal fold micro-tremors in the human voice in the 8-12 Hz range [16]. The state of art in this technology [U.S. Ser. No. 11/441,449; U.S. Pat. No. 7,451,079; U.S. Pat. No. 3,971,034] is based on a computer assisted method of assigning a numeric score to a voice pattern sample of an individual where this score is a kind of measure of the psychological stress of the said human individual. This method is more advantageous because it is not necessary that the individual is physically connected to the device but has the disadvantage of requiring the voluntary speech of a human individual.

Biological markers have also been used as a measure of stress in human individuals. It is well known that stressful events stimulate the human hypothalamus promoting the secretion of corticotrophin hormone (CRH). This CRH starts the Hypothalamic-Pituitary-Adrenal axis resulting in an increased production of cortisol, which can be measured and quantified [17, 18]. Also, the sympathetic nervous system is stimulated when a stressful situation overcomes and this can be measured by the output levels of adrenaline and noradrenaline [17, 18]. The measurements of one or more isoprostanes in a biological sample have been also used as a marker of stress in humans [U.S. Ser. No. 10/660,025; Ref. 19], Isoprostanes can be easily quantified in urine, blood, tears, sweat and saliva, but none of this permit an on-line analysis of the concentrations of the said isoprostanes.

As this moment, there are no known solutions to the problem of (i) detecting stress levels or sudden changes in humor in human or other type of individuals, (ii) in real time or, at least comprising a delay shorter than 30 s, (iii) comprising non-invasively or degrading methods, (iv) not requiring the individual under study to be aware of being studied; and (v) based on the on-line chemical analysis of the composition of the vapors released by the individual through its skin or breath.

### SUMMARY OF THE INVENTION

This invention contributes a new on-line method of detection of stress levels or sudden changes in humor of individuals. The approach is particularly advantageous because the analysis can be performed in real time or, at least, comprising a delay shorter than 30 seconds, and because it is non-invasive and non-degrading and actually the individual under study do not need to be aware of being studied. More specifically, the present invention is based on the analysis of lactic acid, pyruvic acid and other volatiles concentrations in the vapors released by the individual (through the skin and/or the breath). The inventors have proved that said concentrations can response nearly instantaneously when the individual is subjected to sudden stressful stimulus.

### BRIEF DESCRIPTION OF THE DRAWINGS

FIG. 1 shows a XIC of lactic acid (intensity of the signal produced) represented versus time.

FIG. 2 shows a XIC of pyruvic acid (intensity of the signal produced) represented versus time.

FIG. 3 shows a block diagram of the preferred embodiment of the present invention. The main blocks are the sampling step, the dilution step, the ionization step, the analysis step and the signal monitoring step.

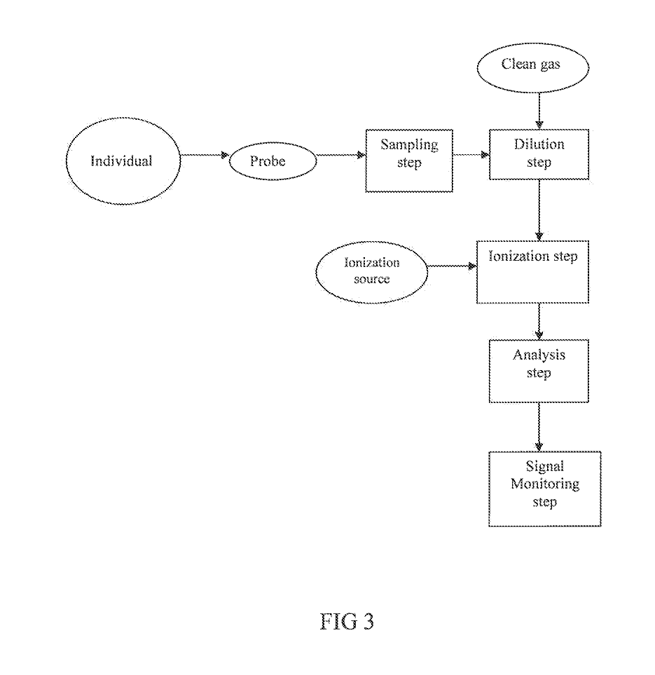

FIG. 4 shows a more detailed diagram of the preferred embodiment of the present invention. The thin arrows show the connections between different blocks, while the hatched arrows show the direction of both the sampled and clean flow towards the “Tee” connection and the ionization chamber.

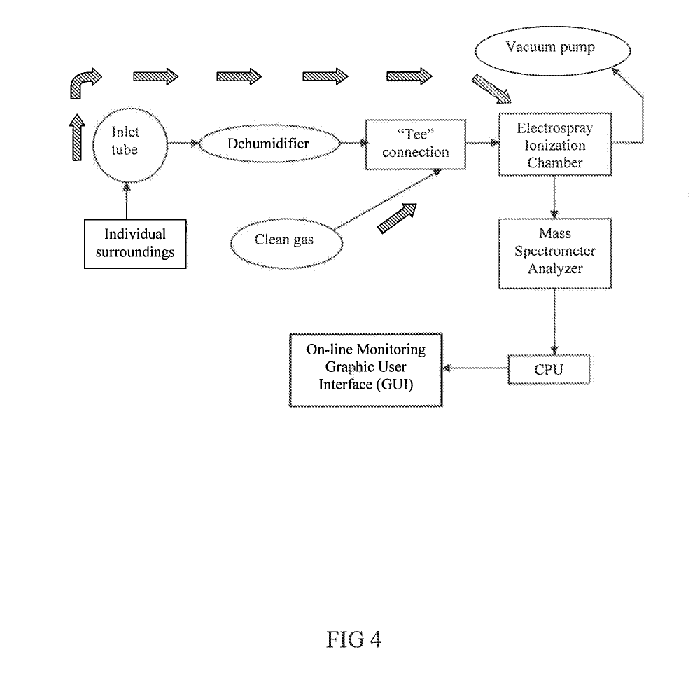

FIG. 5 shows another alternative embodiment of the present invention that comprises two fans, in order to generate a continuous but smooth flow of air from the surroundings of the sampled individual to the collecting probe, thus driving the vapors released by the skin or the breath of the individual, to the ionization chamber.

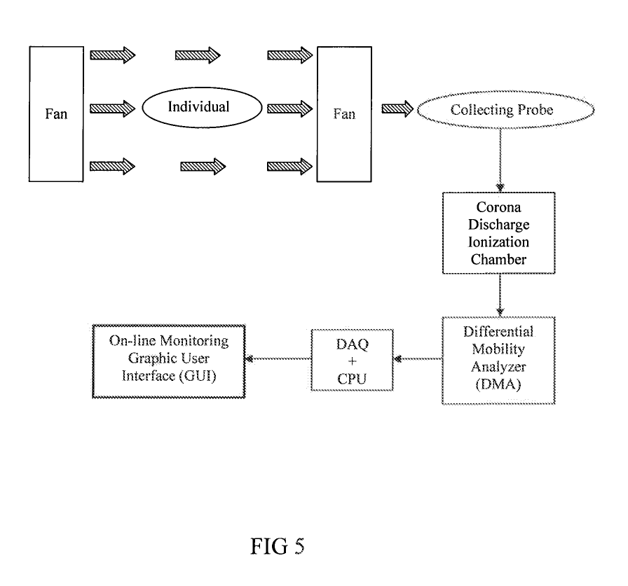

FIG. 6 shows how the individual's sampled vapors enter the ionization chamber in the preferred embodiment of the present invention, are put in contact with an electrospray cloud, become ionized vapors and enter the time-of-flight mass spectrometer ion path to be analyzed.

### MORE DETAILED DESCRIPTION OF THE INVENTION

Target of the invention. The main objective of this invention is to detect or distinguish different stress levels or changes in humor of individuals in real time, or comprising a delay shorter than 30 seconds, neither invasively or being degrading, nor requiring the individual under study to be aware of being studied, by means of on-line analysis of the vapors released by the skin or in breath of the individual.

The signal level of various fatty acids found in the spectra can be used as markers of human metabolism.

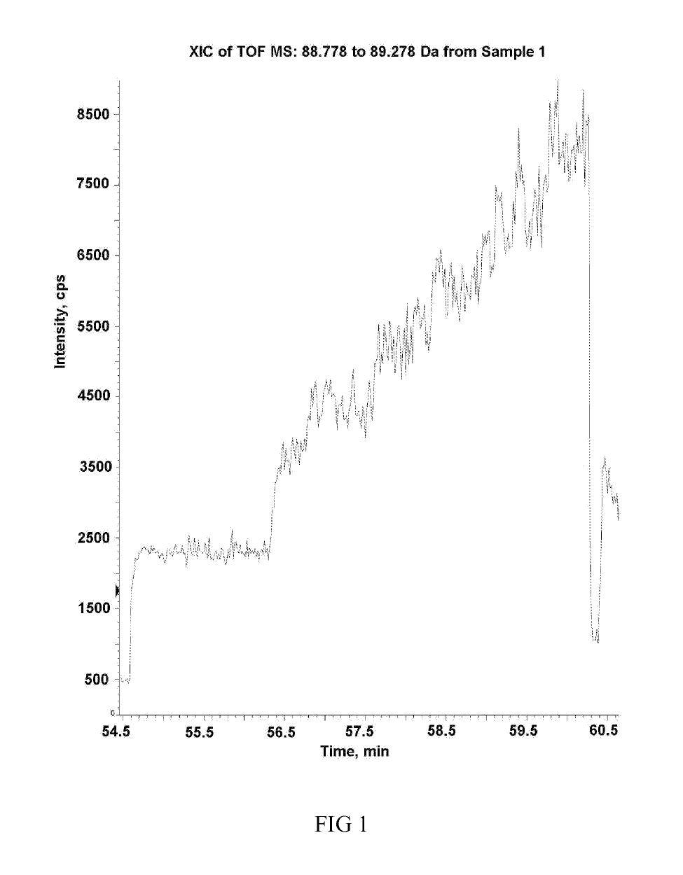

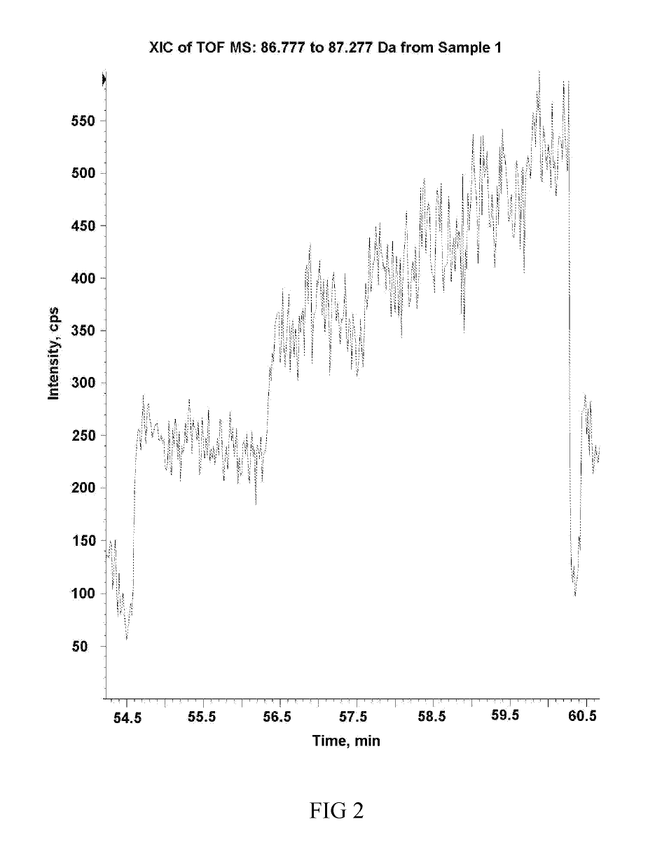

More specifically, the signal level of lactic and pyruvic acids measured at a chemical analyzer is used in this invention as a marker of the individual change in humor or stress level. A sudden change in the individual's humor or stress level is reflected in the lactic and pyruvic acid signal level as a sudden increase with a response time of less than 30 seconds. FIGS. 1 and 2 illustrate the signal level measured by the analyzer.

Description of the method. The method herein described comprises the following steps of (i) sampling vapors released by the skin and/or the breath of the individual; (ii) dilution of individual's vapors to prevent saturation; (iii) ionizating the mixture of vapors; (iv) analyzing the produced ions; and (v) further monitoring of marker species.

(i) Sampling Vapors of the Surrounding Environment of the Individual:

The main objective of this step is to sample the vapors released by the skin or the breath of the individual under study in order to transport them on-line through a tube towards the next steps of this invention. The preferred embodiment of the present invention comprises a tube that captures a continuous flow rate from the surrounding ambient of the individual's extremity, driving it towards the next step of this preferred embodiment. The way the vapors enter this tube is achieved by means of a vacuum pump (or any other way of generating a continuous flux of ambient vapors towards an analyzer) located downstream the analyzer and thus, permitting a continuous flux of gases from the surroundings of the individual to the analyzer. The vapors can also be sampled directly from the breath. The sampling probe can also be placed upon the individual in order to sample the vapors from the convective wake produced by the individual's heat. A smooth continuous flow can be induced in the room where the study is held to produce a controlled wake. The probe has to be placed within the wake of the individual under study. The gas of the room can be controlled and kept clean to keep the background levels of the substances under study as low as possible. The background can be further cleaned by using a probe that touches the skin not allowing contaminated gas from the room to be sampled. For this embodiment, clean gas has to be introduced in the probe to drive the vapors released by the skin and to supply the sampled gas. All those ways of sampling the vapors released by the skin of the individual permit driving the sampled vapors on-line and serve for the purpose of the present invention and therefore they are all part of the present invention. Dehumidifying the sample can be useful especially if the breath is analyzed to avoid any possible interference between water vapor in the breath and the analytical method used. This aspect is also included in the present invention.

(ii) Dilution of sampled vapors: The main objective of this step is to dilute the sampled gas on-line with clean dry gas in order to avoid saturation of the analyzer and to produce an on-line flow of gas at controlled concentration. Note that, if the analyzer is saturated by a too high sample concentration, the changes in vapors concentration (due to said changes of humor) will not cause changes in the signal level produced by the analyzer and therefore they will not be detected. The preferred embodiment of the present invention comprises a ‘tee’ (two inputs and one output) connection in which sampled vapors and clean gas are introduced and mixed. The mixture comprising diluted vapors exits through the ‘tee’ output. Many other ways of mixing the gases are also possible, such as mixing them in an “ad hoc” mixing chamber, and are all part of the present invention.

(iii) Ionization of the mixture of vapors: The main objective of this step is to ionize the sampled vapors on-line in order to detect them in an analyzer such as a MS (mass spectrometer), an IMS (ion mobility spectrometer), or a DMA (differential mobility analyzer). The mixture of clean gas and human vapors sampled are conducted to a vapor ionization chamber through a tube. The preferred embodiment of the present invention comprises an atmospheric electrospray source (ES) which produces a cloud of charged ions and ultra-fine droplets. The charger ions and droplets produced by the ES and the neutral molecules of the sampled vapors are in very intimate contact and thus, said vapors become ionized. This ionization technique was first introduced by John B. Fenn et al, [2] and further developed by P. Martinez-Lozano & J. Fernandez de la Mora [U.S. Ser. No. 11/732,770, Refs. [7], [8]] and G. Vidal [U.S. 61/204,996]. Vapors can also be ionized by radioactive sources, photon ionization, proton transfer, single laser excitation ionization, double laser excitation ionization, etc. All those methods serve for the purpose of ionizing the vapors on-line and thus they are a part of the present invention.

(Iv) Analysis of the produced ions: The main objective of this step is to identify the ionized species and to be able to monitor the signal level of the marker species in real time. The preferred embodiment of the present invention comprises an API-TOF-MS that performs mass/charge analysis of the target species. But other mass or mobility analyzers, such as IMS's or DMA's are valid for the chemical identification of the different species and consequently they are all part of this invention.

(v) Further monitoring of marker species: The main objective of this step is to present the evolution of the markers used to evaluate the changes of humor in real time. These changes of humor can be evaluated through the changes in the level of the marker signal in the analyzer. The marker of the preferred embodiment is lactic acid but this method can be also applied to other volatiles from skin or breath, such as pyruvic acid, that are all part of the present invention. FIGS. 1 and 2 illustrate the evolution of the signal corresponding to lactic and pyruvic acids. The probe is first approached to the individual under study at minute 54.6. At this moment the signal grows because the apparatus is sensing the lactic and pyruvic acid vapor molecules produced by the individual's skin. The signal level before that moment is the background of lactic and pyruvic acid. During the first two minutes the individual was quiet and the signal remains approximately constant because the individual was not experiencing any change of humor. At minute 56,4, the individual was subjected to a sudden stressful stimulus and the signal shows an abrupt increase produced be the increased level of lactic and pyruvic acid vapors released by the individual. At minute 60.3, the probe is finally removed from the individual's surroundings and the signal falls abruptly to the background level. The preferred embodiment of the present invention comprises an on-line monitoring step of the MC signal of lactic acid, as above described for FIG. 1, generated by the detector of a mass spectrometer. However, other signals showing the levels of lactic acid (or other volatiles from skin or breath such as pyruvic acid) in the mixture of vapors, such as signals generated by an electrometer or any other detectors of ions also serve for the purpose of showing the evolution of the signal in real time and are all part of the present invention. This real-time monitoring process is made, in the preferred embodiment of the present invention, using the signal generated by the DAQ system of the mass spectrometer, a CPU unit with input devices (keyboard, mouse, track ball, pads or touch screen), and a GUI to permit the graphical monitoring of the signal.

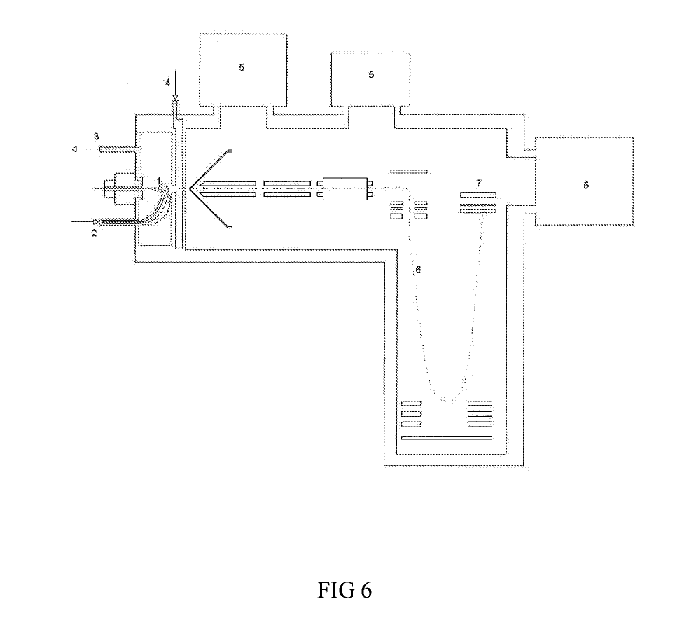

FIG. 6 shows a section view of the ionization chamber and the time-of-flight mass spectrometer used for the preferred embodiment of the present invention. The electrospray cloud (1) is put in contact with the sampled vapors (2) coming from the sampling tube becoming ionized vapors. Then, the ions enter the mass spectrometer ion path and the remaining vapors exit the ionization chamber through an exit tube (3). A counterflow of clean nitrogen (4) is set near the entrance orifice of the mass spectrometer to prevent contamination. The necessary vacuum level for the mass spectrometer is the provided by three turbomolecular pumps (5). The ions are driven through the ion path by means of RF-quadrupoles and then enter the Time of Flight region (6) of the mass spectrometer, to finally hit the detector multi-channel plate thus, producing an electric signal. This signal is then registered through the DAQ system of the mass spectrometer and treated by a CPU to be showed on a computer screen.

What is claimed:

1. A method to detect stress levels or sudden changes of humor in individuals, the method comprising:
sampling the vapors released by the individual into the surrounding air;
analyzing the chemical composition of said vapors by means of an analytical instrument, and
real-time monitoring the signals produced by said analyzer to monitor for stressed dependent species in said vapors.

2. The method of claim 1 where the species monitored are lactic acid or pyruvic acid.

3. The method of claim 1 where the response time of the analyzer is less than 30 seconds.

4. The method of claim 1 where analyzing the sampled vapors comprises the steps of:
introducing said sampled vapors into an ionization chamber where said sampled vapors become ionized vapors;
analyzing said ionized vapors by means of an analytical instrument.

5. The method of claim 4 where the ionization source is an electrospray.

6. The method of claim 4 where said sampled vapors become ionized by a radioactive source creating both positive and negative ions.

7. The method of claim 6 including means to produce electric fields in selected regions of the ionization chamber in order to more efficiently ionize or extract desired ions of a given polarity, such that the ions of the opposite polarity not substantially removed are primarily able to get in contact with said sampled vapors turning them into said ionized vapors.

8. The method of claim 4 where said sampled vapors become ionized by means of a photo-ionization source.

9. The method of claim 4 where said sampled vapors become ionized by means of a corona discharge.

10. The method of claim 4 where said analytical instrument is a mass spectrometer.

11. The method of claim 4 where said analytical instrument distinguishes the various ionized vapors based on their electrical mobility.

12. An apparatus to sample, ionize and analyze neutral vapors from the surroundings of an individual, comprising:
a sampling probe to collect said neutral vapors from the surroundings of said individual;
means to generate a continuous flow of said neutral vapors from the surroundings of said individual towards the analyzer;
an ionization chamber where said sampled vapors become ionized;
an analytical instrument capable of analyzing said ionized vapors in terms of mass to charge relation or mobility;
means to continue real-time monitoring the signal of said ionized vapors in said analytical instrument in order to detect sudden changes of said signals.

13. The apparatus of claim 12 where said ionization process of said sampled vapors is made by one of the following: an electrospray source, a corona discharge, a radioactive source of photons with sufficient energy to produce ions.
